(19) United States
(12) Patent Application Publication
(10) Pub. No.: US 2021/0330302 A1
(43) Pub. Date: Oct. 28, 2021

(54) BIOLOGICAL MATERIAL SAMPLING DEVICE

(71) Applicant: Hoowaki, LLC, Greenville, SC (US)

(72) Inventors: Ralph Hulseman, Greenville, SC (US); Nakul Ravikumar, San Diego, CA (US); Justin Ferdinand, Simpsonville, SC (US); Frank Patterson, Taylors, SC (US); Cameron McPherson, Greenville, SC (US); Andrew Wilkerson, Greenville, SC (US); William Dante Lacher, Greenville, SC (US)

(73) Assignee: Hoowaki, LLC, Greenville, SC (US)

(21) Appl. No.: 17/241,914

(22) Filed: Apr. 27, 2021

Related U.S. Application Data
(60) Provisional application No. 63/015,994, filed on Apr. 27, 2020.

(51) Int. Cl. A61B 10/02

(52) U.S. Cl. CPC A61B 10/02; A61B 2010/0216

(57) ABSTRACT

This specimen collection tool can include a handle including a core manufactured by injecting a first material into a mold; wherein the core includes a flat portion, a protrusion and a proximal portion; a distal end having a cavity for receiving the core manufactured by injecting a second material into the mold wherein the second injection is performed without removing the handle from the mold and the second material surrounds the core; a first set of microfeatures disposed on the distal end having a second set of microfeatures disposed on the first set of microfeatures; a demolding pin included in the distal end; a support surface included on the core to support the core in the mold; and, a tapered tip included in the core operatively associated with a narrowing portion of the distal end to hinder removal of the distal end from the handle.

### RELATED APPLICATIONS

This application is a non-provisional utility patent application claiming the benefit of U.S. provisional patent application Ser. No. 63/015,994 filed Apr. 27, 2020 which is incorporated by reference herein.

### BACKGROUND OF THE INVENTION

### 1. Field of the Invention

This tool is directed to an improved surface for use on articles such as biological specimen collection devices and applicators commonly known as swabs. This tool is also directed for use on biopsy collection devices to replace use of needles, punches and other cutting edge devices.

### 2. Description of the Related Art

The global occurrence of biological specimen collection for diagnostic purposes exceeds 1 billion per year. During a pandemic, it has been determined by health experts that testing, especially rapid, easy-to-use, accurate testing at a national level can assist with reducing the spread of viruses and other diseases by identifying potentially infected individuals leading to those identified individuals to take action or avoid certain actions, both designed to slow or stop the cause of a pandemic. Further, identification of infected individuals can allow these individuals to seek medical attention, isolate themselves, and can allow treatment in advance of serious damage to the infected individual.

Much testing is based upon the process of collecting a biological sample, testing the sample and reporting the results. During the COVID-19 pandemic, it was stated by the New England Journal of Medicine (NEJM) that in regimen of regular testing works as a sort of Covid-19 filter, by identifying, isolating, and thus filtering out currently infected persons, including those who are asymptomatic. Further, the United States Center for Disease Control and Prevention (CDC) estimated in June 2020 that there were 10 times as many Covid-19 cases in the United States as had been detected. The NEJM also stated that for testing to be effective as a pathogen filter that will slow or stop a pandemic, tests are needed to capture most infections while they are still infectious. Such tests can include rapid lateral-flow antigen tests, and rapid lateral-flow tests. The CDC reported in April of 2021 that over 400 million tests were performed in the United States. Other sources have reported that this number is over 430 million. The test performed uses a swab to collect a biological specimen. The CDC recommends that sterile swabs should be used for the collection of upper respiratory specimens. These swabs are important both to ensure patient safety and preserve specimen integrity.

The function of a swab is to collect a biological specimen, store the specimen for transport to a testing location, and then to release the specimen onto media that will be used for analysis. The collection portion of swabs currently produced are made with non-woven fiber bundles, foam, or short fiber flocking. Fiber bundles, foams and flocks negatively interact with the gathered material so that while swabs with this historical technology have been a developed product for some time, there remains a need to improve the effectiveness and ability to collect the specimen. Further, there needs to be an improvement in the amount of specimen that can be stored on a given size swab. Further, there needs to be improvement in the efficiency of release of the specimen to the storage or testing media. Therefore, there is a need for a biological specimen collection device with improved functionality, designed to facilitate increased manufacturing and production, inert, and can accomplish the same or improved biological specimen collection. Notably during periods of great demand such as during disease outbreaks, quickly scalable designs are desirable to meet demand.

It would be advantageous to have a biological collection tool that is effective, patient safe, biocompatible, and simple to make and use, especially for use with high volume testing needs.

### SUMMARY OF THE INVENTION

This invention provides a biological material collection device for the collection, retention and release of a biological sample taken through a passage or orfice from within a patient. The device comprises an elongated shaft with a proximal end and a distal end. The elongated shaft is molded from a female tool cavity to which the molded distal end is then added.

The distal end is comprised of a polymeric material that can be securely bonded to the shaft material. The distal end is molded from a female tool cavity and comprised of a series of biological material collection pillars that are imported from the tool cavity. The distal end can be molded or assembled directly onto the elongated shaft. The molded pillars are designed to perform multiple key performance functions. The pillars' physical properties and geometry present a non-abrasive and non-irritating surface with the patient during insertion of the device and maximally engage the biological material in the collection area. Within the smallest possible diameter, the pillar height and spacing are optimized to collect and retain the biological specimen material. Following removal from the body the pillars allow the specimen to be bathed and efficiently released in the fluid transport media. Critically, the pillars engage, retain, and release RNA in its fullest integrity during the viral specimen collection process.

A specimen collection device for biological material, comprising an elongated shaft including a proximal end portion and distal end portion; a plurality of pillars disposed on the distal end portion of the elongated shaft wherein the plurality of pillars is at least partially circumferentially arranged around the distal end portion of the elongated shaft and configured to engage and retain biological material; wherein the pillars and distal portion are formed from a contiguous and single polymer type. The distal end portion cross-section can be selected from the group of ovoid, circular, planar and in combination. The pillar spacing can be in the range of 5 to 400 microns. The diameter of the pillars can be in the range of 5 to 400 microns. The pillars can have a cross section taken from the group consisting of circular, conical, square, triangular or any combination. The tooling's cross-section can be created with drilling, milling, EDM, or laser cutting. The pillars may have properties taken from the group consisting of a uniform or variable cross-sectional area from 1,800-126,000 μm², a uniform or variable height from 10-1000 μm, an aspect ratio of 0.25 to 6 (height to diameter), uniform or variable spacing, uniform or variable heights, or any combination thereof. The elongated shaft may comprise a thermoplastic or a thermoset polymer. The pillars can be treated with active ligands for selective coupling. The selection of material stiffness and multi-level patterns of pillars seeks to optimize stiffness of pillars for different stiffness tissues and different materials being collected as a function of the stiffness of the tissue and the need for comfort versus the need to remove tissue rather than absorb fluid while collecting material and reducing, minimizing or eliminating trauma. The pillars can be adapted to collect tissue and/or material by wetting, adsorbing, scraping, rasping or any combination thereof.

### BRIEF DESCRIPTION OF THE DRAWINGS

The description of the invention will be explained with reference to the following figures:

FIG. 1A is a front view of aspects of the tool;

FIG. 1B is a front view of aspects of the tool;

FIG. 1C is a front view of aspects of the tool;

FIG. 1D is a cross section of aspects of the tool;

FIG. 1E is a cross section of aspects of the tool;

FIG. 2A is a top view of aspects of the tool;

FIG. 2B is a perspective view of aspects of the tool;

FIG. 2C is a side view of aspects of the tool;

FIG. 2D is a perspective view of aspects of the tool, and

FIG. 3 is an end view of aspects of the tool.

### DESCRIPTION OF THE INVENTION

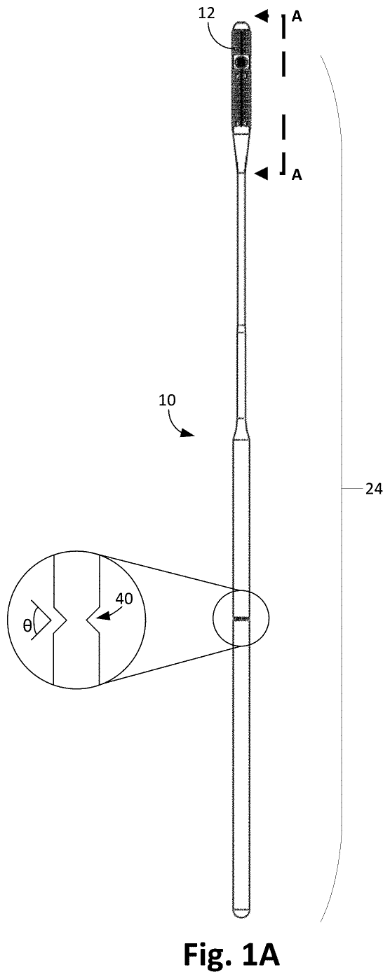

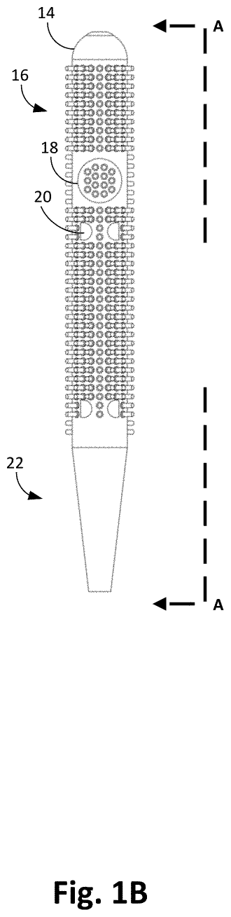

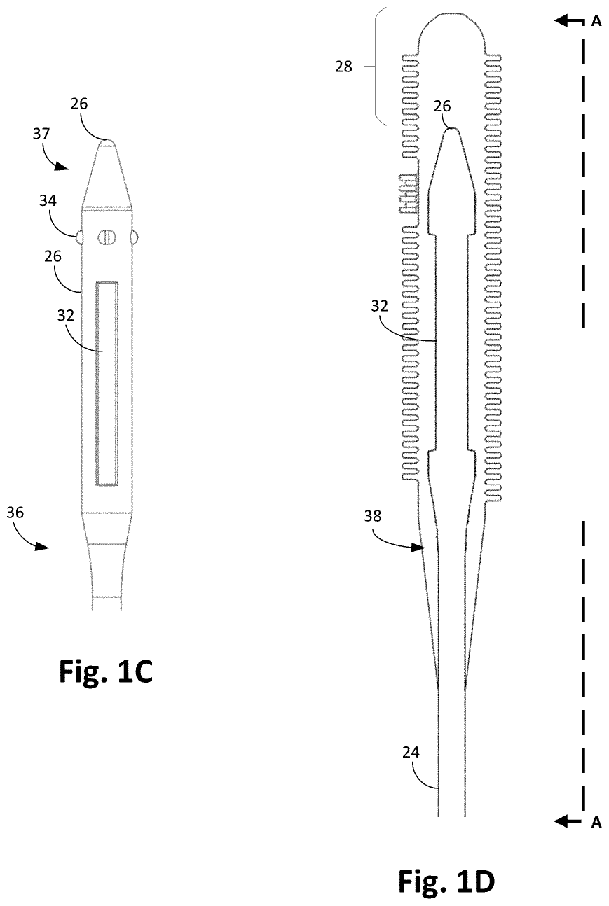

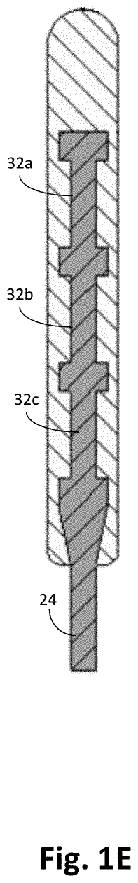

Referring to FIGS. 1A through 1D, a biological collection tool 10 is shown having a distal end 12. Distal end includes section A-A which includes a tip 14, a set of microstructures 16, a de-molding pins 18, support surface 20 and a tapered portion 22. The distal end can be made from a flexible or semi-flexible material such as silicone that is resilient so that when pressure is applied to the distal end, it can compress to provide for more comfort to the patient. The tip can be devoid of microstructures which can reduce the complexity of manufacturing, especially when demolding the tool. Further, the tip devoid of microstructures can reduce the risk of puncturing tissue as the tip can be a smooth surface rather than having protrusions or other features.

The distal end can have a diameter in the range of 1 mm of 8 mm and can be in the range of 3 mm to 5 mm in one embodiment. The length of the distal end can be in the range of 5 mm to 50 mm and in one embodiment, in the range of 12 mm to 18 mm. The microstructures can be in a pattern that can consist of the 31 pillars to 936 pillars and the pillars can be in the range of 50 microns to 750 microns in width and 300 microns to 900 microns in height. In one embodiment, the number of pillers can be in the range of 156 to 468. The values of these ranges include any value incremental within the range.

In one embodiment, the pitch of the features in the microstructures disposed on the distal end can be in rows longitudinal along the distal end length that are smaller than the pitch between pillars that are circumferential around the distal end. The pillars can have an angle of orientation normal to the substrate (e.g., about 90 degrees) over the substrate. In one embodiment, the pillars can extend from the substrate and may have different angles of orientation so that they converge toward each other or diverge away from each other.

The demolding pins can be used to improve the demolding process allowing the distal end, with the handle 24, to be removed without sticking to the tool. The handle can extend into the distal end and be configured so that a core 26 included in the handle 24 does not extend to the tip of the distal end providing for a distance 28 between a core distal end 30 and the tip 14. The handle can include one or more handle sections wherein each section can have a different diameter. Each handle section can have a smaller diameter relative to the handle section adjacent to it at the proximal end.

The core can include a flat section 32 allowing the distal end to better adhere to the core. One or more flat portions can be defined in the perimeter of the core and in one embodiment, can be disposed on opposite sides of the core or can be evenly spaced from each other around the core. Multiple flat sections 32a, 32b and 32c can be defined in the core to improve the distal end adhering to the core.

The core can include protrusions 34 allowing the distal end to better adhere to the core. The one or more protrusions can extend from the perimeter of the core and in one embodiment, can be evenly spaced from each other and can be disposed along the same cross section of the core. The flat portion or the protrusions are beneficial as they can provide for the handle to be molding in a first step and the distal end to the molded in a second step without the handle being removed from the mold.

The core can include a proximal tapered portion 36 which can provide resistance against the distal end when an attempt to pull the distal end away from the core occurs. The proximal tapered portion can be operatively associated with a distal end narrowing portion 38 so than when the distal end if attempted to be pulled from the core, the proximal tapered portion of the core attempts to expand the distal end narrowing portion providing resistance and hindering or preventing the distal end from being removed from the core. The core can include a tapered tip 37.

In one test, the handle having the tapered tip was tested, the distal end was bent back 180 degrees so that the tip of the distal end touched the side of the handle just below the distal end. The handle was inspected and if there was no fracture, full or partial, the distal end and handle was tested again, to the opposite side. The distal end and handle were tested to failure. In three trials, the number of iterations for a tapered tip included in the core was 38, 43 and 34, respectively. Without the tapered tip included in the core (using a rounded core tip), the handle failed on the first iteration. The tapered tip can reduce the stress concentration on the handle, allowing the tip to bend multiple times before breaking. The tapered tip of the core also assists with preventing displacement of the distal end material in the second injection and promotes material of the distal end flowing into the mold.

In one test, the handle is pulled from the distal end to determine the force needed to separate the distal end from the handle. The distal end is secured at its tip and along the handle's axis. The handle is secured, and the distal end pulled away from the handle. The force required to remove the distal end from the handle in three trials was measured to be 3,046; 3,837; and 3,158 grams for a core with a tapered tip. For a core with a rounded tip, the force measured for three trials was found to be 1,558; 1,823; and 1,664 grams. The design of the core can provide for more surface area contact between the core and the distal end thereby providing for improved adhesion between the handle and the distal end without having to remove the handle from a mold to attach the distal end.

During the manufacturing process, the first step can include injecting a rigid or -semi-rigid material into the mold to form the handle including the core. The second step can include injecting a flexible or semi-flexible material into the mold so that the distal end is formed around the core and can adhere to the core. By manufacturing the tool in one step, the production rate of biological collection tools is increased as opposed to the traditional process of making a handle, removing a handle from a mold, preparing flock, and adhering the flock to the end of the handle. The improvement in the constructions, structure and function of this tool not only improve the functionality of the tool itself but improve the manufacturing process and lower costs of making the tool.

The support surface can support the core and prevent the core from becoming out of alignment. If the core becomes out of alignment, the core can curve resulting in a portion of the core protruding from the distal end. If a portion of the core protrudes from the distal end, the formation of the microstructures formed on the distal end is hampered and even prevented for some portion of the distal end resulting in a less effective biological collection tool. Further, to prevent the core from being out of alignment also can prevent the distal from completely adhering to the core and therefore handle.

In one embodiment, the core distal end extends past the demolding pins so that when force is placed on the demolding pins to remove the tool from the mold, the force is aligned to the distal end and core, and therefore the handle, improving the demolding process. When the core is out of alignment, the force applied to the tool during demolding may not be aligned to the core and results in a less efficient demolding process.

The handle can include a break point 40 so that when the lower portion of the handle is bent past a predetermined angle θ, the lower portion of the handle is released from the upper portion of the handle. This feature provides for additional safety to the patient and if the tool is inserted or pressed into or onto the patient past the pressure needed for release, the force on the patient is removed so that this release can prevent injury to the patient.

The microstructure disposed on the distal end can be configured to store material with better performance and functionality than that of a fiber bundle, foam, or flock. Further, the present structure can prevent, reduce, or eliminate wicking which is a disadvantage in the current technology. The dimensions of the micro pattern (e.g., pitch spacing, array configurations, designs, arrangements) can improve fluid pinning and storage and can be modified based upon fluid viscosity of the material to be gathered. The dimensions of the micro pattern can be adapted to improve release of the gathered material or sample.

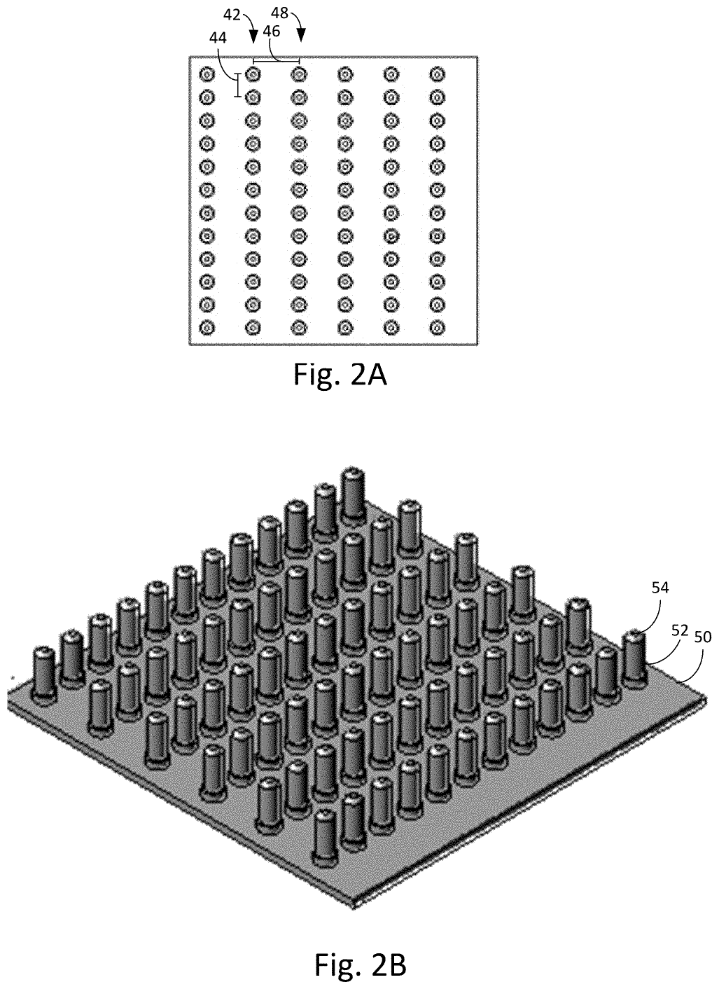

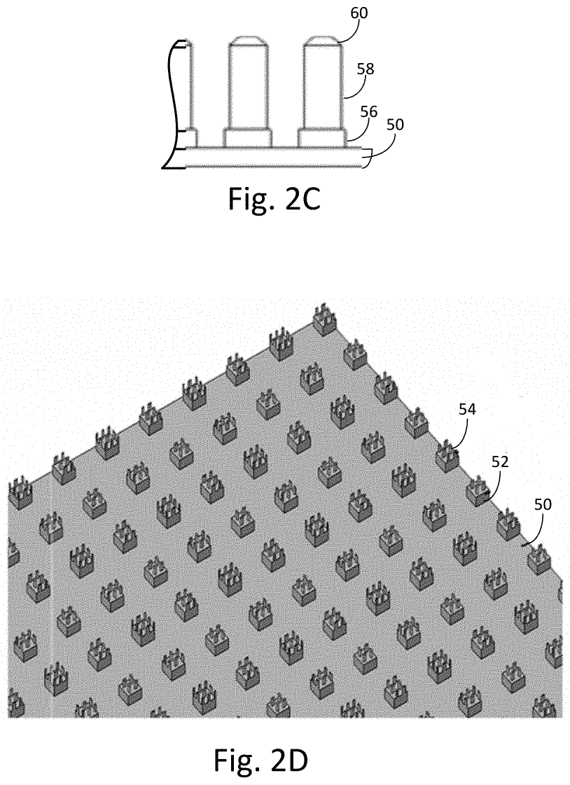

Referring to FIG. 2A, one exemplary microstructure is shown having a first row of pillars 42 having a first spacing 44 between pillars along a first direction a second spacing 46 between adjacent pillars 48 along a second direction. The first spacing can be unequal to the second spacing. The closer spacing can improve the ability to capture a biological sample while the wider spacing can improve the ability to retain the biological sample and can increase the volume of the biological sample collected. The pillars can also be arranged in an off-set configuration wherein the pillars in a row are in a zigzag pattern within a row. The pillars in the row can be unevenly spaced adjacent to each other in the row and non-liner within the row.

Referring to FIGS. 2B and 2D, the microstructure can include two levels of microfeatures. The distal end substrate 50, which can be generally curved so as to partially or completely surround the core, can include a first pillar 52 that can be configured to provide support. A second microfeature 54 can be disposed on the top of the first microfeature to improve the collection of a biological sample. The second microfeature can include multiple pillars so that a pillar in the first microfeature can carry multiple pillars from the second microfeature. The dimensions of the second microfeature can be less than that of the first microfeature in pitch, height, width, and any combination. The second microfeature can be disposed on the top of the first microfeature so that the second microfeature is completed contained on the top of the first microfeature. The substrate 50 can include a first pillar 52 that has a base 56 and extension 58. The top of the extension can include a rounded portion 60. The second microfeature can be disposed on the top of the rounded portion or on the rounded portion itself. The first microfeature can have a height that is greater than the second microfeature. In one embodiment, a third microfeature can be disposed on the side walls of the pillars of the first microfeature.

The first microfeature and the second microfeature can be configured to provide for a fluid-phobic surface, and fluid-phallic surface and any combination thereof. The distal end can include a fluid-phallic portion for receiving and retaining a biological sample and a fluid-phobic surface for improving the release of the biological sample. In one embodiment, the substrate can include a fluid-phobic surface to prevent the biological sample from adhering to the substrate to prevent disadvantageous attachment of the biological sample to the substrate.

The first microfeature and the second microfeature can have the same or differing cross-sections that can be taken from the group consisting of round, oval, square, rectangular, triangular, star, gear shaped, polygon or irregular. The cross-sections can be symmetrical or asymmetrical. Different microfeature pillars on the same object may have different cross-sectional shapes. The first microfeature can have features where one or more of the features possess a different shape, height, pitch, width, and other characteristics. The second microfeature can have features where or more of the features a different shape, height, pitch, width, and other characteristics.

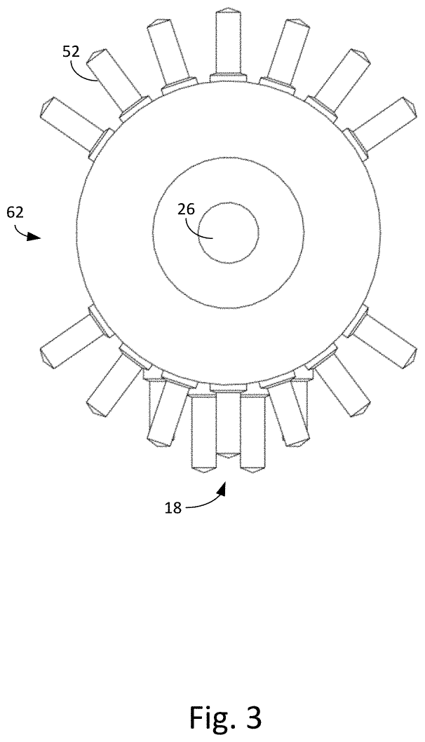

Referring to FIG. 3, the top of the distal end can be seen having tip 26. Pillar 52 of the first microstructures can be seen extending away from the core. The demolding pins 18 can be seen disposed within the microstructures. In one embodiment, clear portions 62 of the distal end can be disposed between microstructures.

### EXAMPLES

The present tool is further described in detail by reference to the following examples. The use of these examples, while used to further describe the tool, would be understood by one skilled in the art not to limit description of the tool, and one skilled in the art would understand that various changes can be made, and equivalents employed, without departing from the scope of the tool.

### Example 1

In one test, a distal end included a microstructure with an array of pillars that have a 400 micron pitch in the longitudinal direction and a pitch varying from 400 microns to 460 microns in the cuircumferential direction. The distal end molded in a female tool using PolyOne VersaFlex™ OM-3060-1 (59 Shore A) rubbery polymer. The tip was demolded and bonded onto a 5.5 inch polystyrene (Styrolution HI 5801 nat) rod. The tip was submerged in water, withdrawn and then examined under magnification for uniform coverage. The water filled uniformly the void areas due to the surface tension created by the pillar design and spacing. The calculated volume of water retention was 75.3 cubic mm or 0.076 grams. A comparative sized recent generation swab made with flock would have water retention of 0.05 grams.

The following illustrates some examples of different microstructure dimensions and properties shown in Table 1.

TABLE 1

| | INDEX | SIZE | | PITCH | | DEPTH |
|---|---|---|---|---|---|---|
| | depth | L1 + L2 + L3 | | L1 + L2 + L3 | | L1 + L2 + L3 |
| Pattern | variant | μm | SHAPE | μm | LATTICE | μm |
| H021 | A | 100 | square | 350 | triangular | 400 |
| H160 | A | 10 + 100 | circle | 20 + 200 | triangular | 20 + 200 |
| | C | 10 + 100 | circle | 20 + 200 | triangular | 20 + 50 |
| H190 | A | 200 | circle | 500 | rectangular | 600 |
| | B | 200 | circle with | 500 | rectangular | 800 |
| | | | tapered base | | | |
| H391 | A | 130 + 200 | circle | 500 | rectangular | 100 + 100 |

### Example 2

A chop force test was used to determine relative hardness of samples when the sample is pushed into the patient and the end overreaches. To perform the test, a razor blade is mounted to a force gauge. The sample is pressed against the razor blade down, perpendicular to the long axis and located over the tip. The force applied when the razor cuts through the entirety of the end is measured. The distal end proved to be easier to slice through due to using softer material resulting in improving patient comfort which correlates to less pain to the patient while the tool is in use. The results as shown in Table 2.

TABLE 2

| | | | | | Standard |
|---|---|---|---|---|---|
| | Trial 1 | Trial 2 | Trial 3 | Average | Deviation |
| Swab Type | (kg) | (kg) | (kg) | (kg) | (kg) |
| Traditional Flock | 4.8 | 4.6 | 4.8 | 4.7 | 0.10 |
| Swab | | | | | |
| Distal End | 1.4 | 1.4 | 1.4 | 1.4 | 0.00 |

### Example 3

Durability and residual effect were tested using an abrasion test. In the test, 60 grit sandpaper was attached to a known weight. While securing one end of the weight to prevent movement of the sandpaper, the samples were dragged underneath the weight thereby contacting the sandpaper. The samples were dragged a set distance. The samples were removed and analyzed for missing collection features. The sandpaper is analyzed for remnants of any features. When features are missing from the sample or remain on the sandpaper, the sample will not continue to be tested. The number of cycles to failure are counted and shown in Table 3.

TABLE 3

| | Trial 1 | Trial 2 | Trial 3 | Average |
|---|---|---|---|---|
| | (cycles | (cycles | (cycles | (cycles |
| Type | to fail) | to fail) | to fail) | to fail) |
| Flock | 1 | 1 | 1 | 1 |
| Foam | 1 | 1 | 1 | 1 |
| Distal End | 4 | 3 | 4 | 3.67 |

The present tool is more durable and are less likely to leave material behind in the patient. The present tool can be used more aggressively to collect a biological sample without risk of failure and with improved comfort to the patient.

### Example 4

A puncture test was performed to determine the comfort of the present tool. This test involves mounting the sample to a force gauge sensor and setting the force gauge to measure maximum compression force. A 1.5-inch by 1.5-inch piece of Syndaver 2N Adult Skin is secured between two square steel frames, each measuring 1.5 inch by 1.5 inch with a thickness of 0.25 inch. The apparatus is placed on a scale and the scale is zeroed for the weight of the apparatus. The scale is set to show peak force. The sample is grasped at the base of the tip and pressed onto and perpendicular to the Syndaver 2N Adult Skin. The peak force reading is taken once the tip pierces the Syndaver 2N Adult Skin. The results are shown in Table 4.

TABLE 4

| | | | | | Standard |
|---|---|---|---|---|---|
| | Trial 1 | Trial 2 | Trial 3 | Average | Deviation |
| Type | (g) | (g) | (g) | (g) | (g) |
| Flock | 1403.78 | 987.84 | 1256.21 | 1215.94 | 210.87 |
| Distal End | 2407.85 | 2257.62 | 2013.94 | 2226.47 | 198.79 |

The present tool is more comfortable for the end user due to the distal end being of a softer material then that of the handle and core. This combination reduces likelihood of injury to the patient during the use of the tool.

### Example 5

A swab deposit test was performed to analyze the ability of a tool to deposit liquid material from the tool to the test media. For this test, the sample is weighted. The sample is dipped into a container of vegetable oil, submerging the entire head of the sample. The sample is removed from the container, allowing all excess to drip off the sample. The sample is weighed again without allowing the sample to come into contact another object or surface. The difference of the weight before and after collection represent the amount of material collected. The sample is then dipped into a container of water and swirled around the perimeter of the container a set number of times, ten in this test. The sample is removed from the water and held horizontally above the container. With the sample over the container, the neck of the sample is tapped on the edge of the container a predetermined number of times, five in this test. The sample is weighed again. The difference of this weight and the weight after collection represent the amount of material deposited. Using all the calculated values of amount collected and deposited, the percent of collected matter deposited can be calculated and shown in Table 5A and Table 5B.

TABLE 5A

| | | | |
|---|---|---|---|
| | Collected | Deposited | Percent (%) |
| Type | (g) | (g) | Deposited |

Trial 1

| | | | |
|---|---|---|---|
| Flock | 0.1356 | 0.0238 | 17.55 |
| Distal End | 0.0534 | 0.0132 | 24.72 |

Trial 2

| | | | |
|---|---|---|---|
| Flock | 0.1318 | 0.0292 | 22.15 |
| Distal End | 0.0389 | 0.0317 | 81.49 |

Trial 3

| | | | |
|---|---|---|---|
| Flock | 0.1397 | 0.0165 | 11.81 |
| Distal End | 0.0339 | 0.0092 | 27.14 |

TABLE 5B

| | Average Amount | Average Percent (%) |
|---|---|---|
| Type | Deposited (g) | Deposited |
| Flock | 0.0232 | 17.17 |
| Distal End | 0.0180 | 44.45 |

The present tool is more efficient in depositing liquid material from the distal end to a test media than the traditional flock based swab.

### Example 6

A retainment test was performed by weighing the sample. Dipping the sample into a container of vegetable oil, submerging the entire head of the sample. Removing the sample from the container, allowing all excess to drip off. The sample is weighed again without allowing the sample tip to come into contact with any object or surface. The difference between this weight and the initial weighing of the sample represents the amount of material collected by the sample. The sample is suspended vertically with the tip pointing downward for a predetermined period of time, one hour in this test. After the predetermined period of time, the sample is weighed. The difference between this weight and the weight after collection represent the amount of material lost as shown in Tables 6A and 6B.

TABLE 6A

| | | | |
|---|---|---|---|
| | Collected | Deposited | Percent (%) |
| Type | (g) | (g) | Deposited |

Trial 1

| | | | |
|---|---|---|---|
| Flock | 0.2108 | 0.1871 | 0.0237 |
| Distal End | 0.0544 | 0.0390 | 0.0154 |

Trial 2

| | | | |
|---|---|---|---|
| Flock | 0.1431 | 0.1021 | 0.0410 |
| Distal End | 0.0377 | 0.0199 | 0.0178 |

Trial 3

| | | | |
|---|---|---|---|
| Flock | 0.2107 | 0.1618 | 0.0489 |
| Distal End | 0.0325 | 0.0214 | 0.0111 |

TABLE 6B

| Type | Average Amount Lost (g) |
|---|---|
| Flock | 0.0379 |
| Distal End | 0.0148 |

The present tool loses less material due to a stronger retention of the liquid.

The handle was tested where the core included the tapered portion.

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art. For example, when a compound is claimed, it should be understood that compounds known in the prior art, including certain compounds disclosed in the references disclosed herein (particularly in referenced patent documents), are not intended to be included in the claim.

The pillars can be treated with active ligands for selective coupling. The selection of material stiffness and multi-level patterns of pillars can be determined to optimize stiffness of pillars for different stiffness tissues and different materials being collected as a function of a stiffness of a tissue and the need for comfort versus the need to remove the tissue rather than absorb fluid while collecting material and reducing, minimizing, or eliminating trauma. The pillars can be adapted to collect tissue and material by wetting, adsorbing, scraping, rasping or any combination thereof.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure.

Every formulation or combination of components described or exemplified can be used to practice the invention, unless otherwise stated. Specific names of materials are intended to be exemplary, as it is known that one of ordinary skilled in the art can name the same material differently. One of ordinary skilled in the art will appreciate the methods, device elements, starting materials, and synthetic methods other than those specifically exemplified can be employed in the practice of the invention without resort to undue experimentation. All art-known functional equivalents, of any such methods, device elements, starting materials, and synthetic methods are intended to be included in this invention. Whenever a range is given in the specification, for example, a temperature range, a time range, or a composition range, all intermediate ranges and subranges, as well as all individual values included in the ranges given, are intended to be included in the disclosure.

As used herein, “comprising” is a synonym for “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A specimen collection tool comprising
a handle including a core configured to be manufactured by injecting a first material into a mold during a first injection;
wherein the core includes a flat portion, a protrusion and a proximal portion;
a distal end having a cavity for receiving the core configured to be manufactured by injecting a second material into the mold during a second injection wherein the second injection is performed without removing the handle from the mold and the second material surrounds the core;
a first set of microfeatures disposed on the distal end having a second set of microfeatures disposed on the first set of microfeatures;
a demolding pin included in the distal end and wherein the core extends into the distal end past the demolding pin;
a support surface included on the core to support the core in the mold and configured to reduce curving of the core during the second injection; and,
a tapered tip included in the core operatively associated with a narrowing portion of the distal end to hinder removal of the distal end from the handle.

2. The specimen collection tool of claim 1 including:
a first row of pillars included in the first set of microfeatures wherein each pillar in the first row of pillars is a first distance from each other, and
a second row of pillars adjacent to the first row of pillars wherein a first pillar in the first row of pillars is a second distance from a second pillar in the second row of pillars and wherein the first distance and the second distance are not equal.

3. The specimen collection tool of claim 1 wherein the support surface is a first support surface and disposed in a middle area of the core and a second support surface included in the core and disposed at a proximal area of the core.

4. The specimen collection tool of claim 1 including a first handle section having a first diameter and a second handle section having a second diameter wherein the first diameter is not equal to the second diameter.

5. The specimen collection tool of claim 1 including a break point defined in the handle.

6. The specimen collection tool of claim 1 wherein the specimen collection tool has over three times a cycle rate to failure when compared to a flock swab and a foam swab using an abrasion test.

7. The specimen collection tool of claim 1 wherein the distal end includes a tip devoid of microstructures.

8. A specimen collection tool comprising
a handle including a core configured to be manufactured by injecting a first material into a mold during a first injection;
a distal end having a cavity for receiving the core configured to be manufactured by injecting a second material into the mold during a second injection wherein the second injection is performed without removing the handle from the mold;
a first set of microfeatures disposed on the distal end having a second set of microfeatures disposed on a top of the first set of microfeatures; and,
a tapered tip included in the core operatively associated with a narrowing portion of the distal end to hinder removal of the distal end from the handle.

9. The specimen collection tool of claim 8 including a demolding area included in the distal end.

10. The specimen collection tool of claim 9 including a demolding pin disposed in the demolding area.

11. The specimen collection tool of claim 9 wherein the core extends into the distal end past the demolding area.

12. The specimen collection tool of claim 8 including a support surface included on the core to support the core in the mold.

13. The specimen collection tool of claim 12 wherein the support surface is configured to reduce curving of the core during the second injection.

14. The specimen collection tool of claim 8 including:
a first row of pillars included in the first set of microfeatures wherein each pillar in the first row of pillars is a first distance from each other, and
a second row of pillars adjacent to the first row of pillars wherein a first pillar in the first row of pillars is a second distance from a second pillar in the second row of pillars and wherein the first distance and the second distance are not equal.

15. The specimen collection tool of claim 8 wherein the first material has a higher rigidity than the second material.

16. The specimen collection tool of claim 8 wherein the narrowing portion of the distal end is formed by the second injection.

17. A specimen collection tool comprising
a handle including a core configured to be manufactured by injecting a first material into a mold during a first injection;
a distal end manufactured around the core by injecting a second material into the mold during a second injection wherein the second injection is performed without removing the handle from the mold; and,
a set of microfeatures disposed on the distal end configured to receive a material and deposit the material on a test media.

18. The specimen collection tool of claim 17 including a tapered tip formed on the core during the first injection and a narrowing portion formed in the distal end during the second injection.

19. The specimen collection tool of claim 17 including:
a first row of pillars included in the set of microfeatures wherein each pillar in the first row of pillars is a first distance from an adjacent pillar, and
a second row of pillars adjacent to the first row of pillars wherein a first pillar in the first row of pillars is a second distance from a second pillar in the second row of pillars and wherein the first distance and the second distance are not equal.

20. The specimen collection tool of claim 17 wherein the set of microfeatures are disposed on a substrate included in the distal end and the substrate is fluid phobic.
